(19) United States
(12) Patent Application Publication
(10) Pub. No.: US 2007/0043263 A1
(43) Pub. Date: Feb. 22, 2007

(54) Simultaneous magnetic control of multiple objects

(76) Inventor: Glenn Mark Wakefield, Tempe, AZ (US)

Correspondence Address: Glenn Wakefield, 1416 East Carmen Street, Tempe, AZ, 85283-4142, US

(21) Appl. No.: 11/586,098

(22) Filed: Oct. 25, 2006

Related U.S. Application Data
(63) Continuation-in-part of application No. 10/406,336, filed on Apr. 3, 2003, now abandoned.
(60) Provisional application No. 60/731,645, filed on Oct. 29, 2005.

(51) Int. Cl. A61B 1/00; A61B 5/05

(52) U.S. Cl. 600/160; 600/117; 600/407

(57) ABSTRACT

A magnetic system controlling one or more objects that may be used in medical applications, industrial applications and construction applications. The applications will be of any complexity and range from the arbitrarily small to the arbitrarily large. The objects may have physical connections among themselves and to the main unit

### CROSS-REFERENCE TO RELATED APPLICATIONS

Nonprovisional application No. 10/406336

Provisional application No. 60/731,645

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISK

Not Applicable

### BACKGROUND OF THE INVENTION

1. Field of the Invention

This patent applies to the field of magnetic control systems.

2. Background Art

An external magnetic field that controls a capsule has been proposed for the medical examination of the gastrointestinal tract. This system is limited to the control of one capsule for a simplistic examination of the gastrointestinal tract.

### BRIEF SUMMARY OF THE INVENTION

A magnetic system controlling one or more objects that may be used in medical applications, industrial applications and construction applications. The applications will be of any complexity and range from the arbitrarily small to the arbitrarily large. The objects may have physical connections among themselves and to the main unit.

### BRIEF DESCRIPTION OF THE SEVERAL VIEWS OF THE DRAWING

Not applicable

### DETAILED DESCRIPTION OF THE INVENTION

This design assumes the information expressed in the patents filed at the USPTO in April 2003 10/406,336 and at the PCT/USPTO in March 2004 under the title “Magnetically Propelled Capsule Endoscopy” and the provisional patent 60/731,645. This information will be treated as if it is typed here.

A magnetically responsive object of arbitrary shape has its movement controlled by an external magnetic field. One or more of these magnetically responsive objects may be simultaneously controlled, working in synchronicity on a common objective or working independently. This is made possible by an appropriate number of magnetic field generating structures. A magnetic field generating structure of any appropriate design will produce the required magnetic field. Each magnetically responsive object will be composed of any instruments, any electromechanical objects and zero, one or more physical connections to other magnetically responsive objects or to other objects in the system. All of the information in this patent will apply as appropriate to magnetically responsive objects with zero, one or more external physical connections. The physical connections may be limited to connections among the magnetically responsive objects with no connection to the external control unit.

The system can be implemented in medical applications, industrial applications and construction applications. The applications will be of any complexity and will range from the arbitrarily small to the arbitrarily large. The procedures performed by the system may be as diverse as a complex surgical procedure to the machining of the inside of a long tube to exact contour and tolerance to the construction of a tiny electromechanical system that is not visible to the naked eye. The magnetically responsive objects may be programmed to carry out procedures or to be operated manually in real time. An example of a surgical procedure is one or more magnetically responsive objects coordinating for a procedure on the gastrointestinal tract. Everything would be computer controlled to minimize contact with the intestine and to allow for efficient viewing and operational procedures. There would be human override capability. This would enable procedures to be done on the entire intestinal tract or on any system within the human body or other animal with no or minimal external incision marks. All of the magnetically responsive objects will be appropriately designed for the individual environments in which they will be functioning.

Every aspect of the entire system will be under a closed loop control system, an open loop control system or an appropriate combination. This will cover everything including 1.) the magnetic field generating structures, 2.) all components associated with the magnetically responsive objects [magnetically responsive elements; illumination generation; illumination detection; optics; communication; navigation (sensors—orientation, position, velocity, acceleration, jerk, magnetic field intensity and direction); mechanical and electrical devices; instruments; power source; electronics], 3.) the movement and stabilization of all magnetically responsive objects relative to each other and the surroundings, 4.) physical connections between and or among magnetically responsive objects and between and or among magnetically responsive objects and other structures (for example suction and or pressure hoses for gas, liquid, solid, plasma, elementary particles; communications; power; fiber laser or other type of laser), 5.) electrical shock protection system by sensitive ground fault and arc fault systems of appropriate response time or by any other appropriate electrical shock protection system. Any information generated by any part of the system may be used by any other portion of the system for control purposes and for any other appropriate purpose.

One type of electrical shock protection along any external physical connection employs the following method: 1.) an outside covering of each appropriate external physical connection and or an overall outside protective covering of all external physical connections, 2.) the covering consists of at least five layers insulator-conductor-insulator-conductor-insulator with a varying or constant potential difference between the conductors, 3.) if the covering is penetrated a monitored circuit will detect the interference with the layered structure and protection circuitry will be activated. This alternating layer concept may be generalized to any method of detecting penetration of the protective cover. This type protection system may be appropriately applied to the magnetically responsive objects or other parts of the system or to any product on the market in general.

Piezoelectric strand like projections (or any other appropriate arrangement—for example flush) and or regular pressure transducers may be strategically located on the magnetically responsive objects, on the external physical connections and on any mechanical and electrical devices. The piezoelectric structures and regular transducers will detect movement and pressure against the surroundings. The piezoelectric structures and regular transducers will be appropriately coated and electrically connected up.

All magnetically responsive objects will be run by an internal computer (DSPs, microcontrollers, microprocessors). All external magnetic field generating structures and other system components will be run by a computer (DSPs, microcontrollers, microprocessors). The magnetically responsive objects will vary in size and function which will include the use of different magnetic field generating structures (for example size, magnetic intensity range, response time).

There are several approaches to simultaneously controlling one or more magnetically responsive objects. The rectangular cube may have magnetic field generating devices on each face. Each rectangular cube of arbitrary size will control one magnetically responsive object. By physically moving the field generator along with varying its power input (for example current) the magnetically responsive objects will be moved and stabilized. For example in a gastrointestinal exam, the field generators will follow the magnetically responsive objects down the chest and into the abdomen. The imaginary rectangular cube may be deformed to any appropriate shape as it is all under computer control. This will permit the field generators to be in very close proximity. This will allow many magnetically responsive objects to be controlled with minimal interference among the field generators. Appropriate tradeoffs between precision control and the number of controlled magnetically responsive objects may be varied in real time.

The term magnet applies to any type of magnet, magnetically responsive substance or magnetically generating object which includes electromagnets with or without a core, anisotropic magnets, isotropic magnets, magnetic substance, diamagnetic substance, paramagnetic substance, ferromagnetic substance and electromagnetic generating object. Assume each magnetically responsive object is a three dimensional ellipsoid for explanation purposes. The ellipsoid will have a rectangular coordinate system with the point (0,0,0) located at the center of the ellipsoid. A pair of magnets with each magnet located near the opposite ends of an axis will allow each magnetically responsive object to be effectively controlled. The magnetic field may be symmetric with the axis. There may be three pairs of magnets with each pair of magnets correspondingly associated with each axis of the rectangular coordinate system. In place of two magnets associated with each axis, there may be four (lying on the vertices of a rectangle associated with each axis) or eight magnets (lying on the vertices of a rectangular cube associated with each axis). In general one or more magnets may be associated with one or more axes with the magnets located to permit all of the other functions of the magnetically responsive objects to efficiently carried out. The magnets associated with each axis may be distributed as necessary for optimal control. One or more axes with an appropriate magnet distribution will provide a varying degree of control. The magnets may be located on only one end of the axis as necessary (for example corresponding to a three dimensional polyhedra with non parallel faces or other general shapes). The magnets may be spaced evenly across the entire two dimensional surface of the ellipsoid providing for smoother functioning in appropriate cases.

If electromagnets or magnetic elements that may be controlled in real time are used in the magnetically responsive objects, the external field generators may be reduced to one or two (one on each opposing face of a cube). Good control will be maintained in this situation. The two magnetic field generating structures and the magnetic field produced by an appropriate configuration of magnets internal to the magnetically responsive objects will have to be controlled in real time. By varying the magnetic field of the internal magnets many more magnetically responsive objects will be simultaneously controllable with minimal interference among the magnetic fields. In this situation one or more field generators would provide varying degrees of control. The appropriate power conserving and switching circuitry among the electromagnets in the magnetically responsive objects will provide for efficient operation.

If for movement, stabilization and procedure purposes one or more magnetically responsive objects make use of internal electromagnets or magnetic elements that are controllable in real time via the direction of the field, then a safe, sufficient power source is necessary. With an external physical connection a magnetically responsive object may obtain power from a direct wire connection that is appropriately protected. Other options include an external light source—laser, maser or otherwise—impinging on a solar cell, thermoelectric device or other appropriate electromagnetic wave gathering device within the magnetically responsive object. An additional possibility is an external air source to power an air electrical generator within the magnetically responsive object. Appropriate flow, direction control and storage (energy storage) for the air will be provided for by electronically controlled valves. Without a physical connection a magnetically responsive object may be powered by an arbitrarily small to arbitrarily large engine with an appropriate fuel supply, oxidizer supply and exhaust system. Other options include an external light source—laser, maser or otherwise—impinging on a solar cell, thermoelectric device or other appropriate electromagnetic wave gathering device within the magnetically responsive object. Where appropriate an electrical protection system is required. Instead of the internal electromagnets rotating the magnetically responsive object, the components as appropriate except the electromagnets rotate into position. An appropriate combination of both internal electromagnet rotation and internal component rotation will provide a more optimized solution.

In all cases of controlling one or more magnetically responsive objects with one or more field generators, a tightly coupled control system among the internal and external magnets (sensors—pressure, position, velocity, acceleration, jerk, orientation, field strength and direction, magnetic permeability of all system components and surroundings) is mandatory. A real time electromagnetic field solver is necessary for optimal control.

Magnetically responsive objects with an ultrasound instrument may interface with one or more external ultrasound devices in a real time control system to produce a high quality image of the examined structure. Ultrasound may be replaced with a magnetic field or any of the other emitting and detecting technologies listed above. These systems may be used simultaneously as appropriate.

I claim:

1. A system composed of one or more external magnetic field generators for controlling:
one or more magnetically responsive objects with an external physical connection in which the fields of the external magnetic generators, the internal magnetic elements or both will be varied in real time;
one or more magnetically responsive objects without an external physical connection in which the fields of just the internal magnetic elements or both the external magnetic generators and the internal magnetic elements will be varied in real time;
two or more magnetically responsive objects without an external physical connection in which the fields of external magnetic generators, the internal magnetic elements or both will be varied in real time;
combinations of the above.

2. Referring to claim 1, every aspect of the entire computer controlled system will be under a closed loop control system, an open loop control system or an appropriate combination in which the information generated by any part of the system may be used by any other portion of the system for control purposes or any other appropriate purpose.

3. Referring to claim 1, the term magnet or magnetic will refer to any type of magnet, magnetically responsive substance or magnetically generating object which includes electromagnets with or without a core, anisotropic magnets, isotropic magnets, magnetic substance, diamagnetic substance, paramagnetic substance, ferromagnetic substance and electromagnetic generating objects.

4. Referring to claim 1, the arrangement of one or more magnets in the magnetically responsive object will be optimized depending on the design criteria.

5. Referring to claim 1, the system can be implemented in medical applications, industrial applications and construction applications in which the applications will be of any complexity and will range from the arbitrarily small to the arbitrarily large and include procedures as diverse as a complex surgical procedure to the machining of an arbitrary part to exact contour and tolerance to assembly line production to the construction of a tiny electromechanical system that is not visible to the naked eye.

6. Referring to claim 1, a real time electromagnetic field solver is necessary for optimal control since a tightly coupled control system among the internal and external magnets (sensors—pressure, position, velocity, acceleration, jerk, orientation, field strength and direction, magnetic permeability of all system components and surroundings) is mandatory.

7. Referring to claim 1, an electrical fault prevention system is necessary in appropriate situations.

8. Referring to claim 1, the term physical connections refers to one or more physical connections between and or among magnetically responsive objects and between and or among magnetically responsive objects and other structures (for example suction and or pressure hoses for gas, liquid, solid, plasma, elementary particles; communications; power; fiber laser or other type of laser).

9. Referring to claim 1, one or more magnetically responsive objects with ultrasound instruments, electromagnetic field instruments and or elementary particle instruments may interface with one or more external instruments of a compatible nature to produce a high quality image of the examined structure.

10. Referring to claim 1, piezoelectric strand like elements and normal pressure transducers are appropriately attached to the magnetically responsive object and components thereof and to the physical connections to detect contact with the external environment.

11. Referring to claim 1, a power source for magnetically responsive objects which have an external physical connection include a direct wire connection, an external light source (laser, maser or otherwise) impinging on a solar cell, thermoelectric device or other appropriate electromagnetic wave gathering device within the magnetically responsive object and an external air source to power an air electrical generator within the magnetically responsive object.

12. Referring to claim 1, a power source for magnetically responsive objects which do not have an external physical connection include an arbitrarily small to arbitrarily large engine with an appropriate fuel supply, oxidizer supply and exhaust system and an external light source (laser, maser or otherwise) impinging on a solar cell, thermoelectric device or other appropriate electromagnetic wave gathering device within the magnetically responsive object

13. Referring to claim 1, instead of the internal electromagnets rotating the magnetically responsive object, the internal components as appropriate (except the electromagnets) will rotate into position or a combination of both internal electromagnet rotation and internal component rotation as appropriate will provide a more optimized solution.
